(19) United States
(12) Patent Application Publication
(10) Pub. No.: US 2019/0328832 A1
(43) Pub. Date: Oct. 31, 2019

(54) ANTIMICROBIAL COMBINATIONS

(71) Applicant: HELPERBY THERAPEUTICS LIMITED, London (GB)

(72) Inventors: Anthony COATES, London (GB); Yanmin HU, London (GB)

(21) Appl. No.: 16/334,003

(22) PCT Filed: Sep. 18, 2017
(86) PCT No.: PCT/GB2017/052752

§ 371 Date: Mar. 16, 2019

(30) Foreign Application Priority Data
Sep. 20, 2016 (GB) 1616014.5

(51) Int. Cl. A61K 38/12; A61K 31/549; A61K 31/5415; A61K 31/437; A61K 31/166; A61K 31/135; A61K 31/4535; A61P 31/04

(52) U.S. Cl. CPC A61K 38/12; A61K 31/549; A61K 31/5415; A61P 31/04; A61K 31/166; A61K 31/135; A61K 31/4535; A61K 31/437

(57) ABSTRACT

The invention provides a combination comprising at least one compound selected from metergoline, proglumide, sertraline, hydroflumethiazide, perphenazine, fluphenazine and pimethixene, or a pharmaceutically acceptable derivative thereof, and a polymyxin selected from polymyxin B and colistin, or a pharmaceutically acceptable derivative thereof. This combination is particularly useful for the treatment of microbial infections.

### CROSS-REFERENCE TO RELATED APPLICATION

This application is a 35 U.S.C. § 371 national stage filing of PCT Application No. PCT/GB2017/052752 filed on Sep. 18, 2017, which claims priority to Great Britain Patent Application No. 1616014.5 filed on Sep. 20, 2016, each of which are incorporated herein in their entirety by reference.

### FIELD OF THE INVENTION

The present invention relates to the combination of known compounds or pharmaceutically acceptable derivatives thereof with a polymyxin selected from colistin and polymyxin B or a pharmaceutically acceptable derivative thereof, and the use of such combinations for the treatment of microbial infections. In particular, it relates to the use of such combinations to kill multiplying microorganisms associated with microbial infections. The known compounds are selected from the group consisting of metergoline, proglumide, sertraline, hydroflumethiazide, perphenazine, fluphenazine and pimethixene or a pharmaceutically acceptable derivative thereof.

### BACKGROUND

Before the introduction of antibiotics, patients suffering from acute microbial infections (e.g. tuberculosis or pneumonia) had a low chance of survival. For example, mortality from tuberculosis was around 50%. Although the introduction of antimicrobial agents in the 1940s and 1950s rapidly changed this picture, bacteria have responded by progressively gaining resistance to commonly used antibiotics. Now, every country in the world has antibiotic-resistant bacteria. Indeed, more than 70% of bacteria that give rise to hospital acquired infections in the USA resist at least one of the main antimicrobial agents that are typically used to fight infection (Nature Reviews, Drug Discovery, 1, 895-910 (2002)).

One way of tackling the growing problem of resistant bacteria is the development of new classes of antimicrobial agents. However, until the introduction of linezolid in 2000, there had been no new class of antibiotic marketed for over 37 years. Moreover, even the development of new classes of antibiotic provides only a temporary solution, and indeed there are already reports of resistance of certain bacteria to linezolid (Lancet, 357, 1179 (2001) and Lancet, 358, 207-208 (2001)).

In order to develop more long-term solutions to the problem of bacterial resistance, it is clear that alternative approaches are required. One such alternative approach is to minimize, as much as is possible, the opportunities that bacteria are given for developing resistance to important antibiotics. Thus, strategies that can be adopted include limiting the use of antibiotics for the treatment of non-acute infections, as well as controlling which antibiotics are fed to animals in order to promote growth. However, in order to tackle the problem more effectively, it is necessary to gain an understanding of the actual mechanisms by which bacteria generate resistance to antibiotic agents. To do this requires first a consideration of how current antibiotic agents work to kill bacteria.

Antimicrobial agents target essential components of bacterial metabolism. For example, the β-lactams (e.g. penicillins and cephalosporins) inhibit cell wall synthesis, whereas other agents inhibit a diverse range of targets, such as DNA gyrase (quinolones) and protein synthesis (e.g. macrolides, aminoglycosides, tetracyclines and oxazolidinones). The range of organisms against which the antimicrobial agents are effective varies, depending upon which organisms are heavily reliant upon the metabolic step(s) that is/are inhibited. Further, the effect upon bacteria can vary from a mere inhibition of growth (i.e. a bacteriostatic effect, as seen with agents such as the tetracyclines) to full killing (i.e. a bactericidal effect, as seen, e.g. with penicillin).

Bacteria have been growing on Earth for more than 3 billion years and, in that time, have needed to respond to vast numbers of environmental stresses. It is therefore perhaps not surprising that bacteria have developed a seemingly inexhaustible variety of mechanisms by which they can respond to the metabolic stresses imposed upon them by antibiotic agents. Indeed, mechanisms by which the bacteria can generate resistance include strategies as diverse as inactivation of the drug, modification of the site of action, modification of the permeability of the cell wall, overproduction of the target enzyme and bypass of the inhibited steps. Nevertheless, the rate of resistance emerges to a particular agent has been observed to vary widely, depending upon factors such as the agent's mechanism of action, whether the agent's mode of killing is time- or concentration-dependent, the potency against the population of bacteria and the magnitude and duration of the available serum concentration.

It has been proposed (Science, 264, 388-393 (1994)) that agents that target single enzymes (e.g. rifampicin) are the most prone to the development of resistance. Further, the longer those suboptimal levels of antimicrobial agent are in contact with the bacteria, the more likely the emergence of resistance.

Moreover, it is now known that many microbial infections include sub-populations of bacteria that are phenotypically resistant to antimicrobials (J. Antimicrob. Chemother., 4, 395-404 (1988); J. Med. Microbiol., 38, 197-202 (1993); J. Bacteriol., 182, 1794-1801 (2000); ibid. 182, 6358-6365 (2000); ibid. 183, 6746-6751 (2001); FEMS Microbiol. Lett., 202, 59-65 (2001); and Trends in Microbiology, 13, 34-40 (2005)). There appear to be several types of such phenotypically resistant bacteria, including persistant, stationary-phase bacteria, as well as those in the depths of biofilms. However, each of these types is characterised by its low rate of growth compared to log-phase bacteria under the same conditions. Nutritional starvation and high cell densities are also common characteristics of such bacteria.

Although resistant to antimicrobial agents in their slow-growing state, phenotypically resistant bacteria differ from those that are genotypically resistant in that they regain their susceptibility to antimicrobials when they return to a fast-growing state (e.g. when nutrients become more readily available to them).

The presence of phenotypically resistant bacteria in an infection leads to the need for prolonged courses of antimicrobial agents, comprising multiple doses. This is because the resistant, slowly multiplying bacteria provide a pool of “latent” organisms that can convert to a fast-growing state when the conditions allow (thereby effectively re-initiating the infection). Multiple doses over time deal with this issue by gradually killing off the “latent” bacteria that convert to “active” form.

However, dealing with “latent” bacteria by administering prolonged courses of antimicrobials poses its own problems. Prolonged exposure of bacteria to suboptimal concentrations of antimicrobial agent can lead to the emergence of genotypically resistant bacteria, which can then multiply rapidly in the presence of even high concentrations of the antimicrobial.

Long courses of antimicrobials are more likely to encourage the emergence of genotypic resistance than shorter courses on the grounds that non-multiplying bacterial will tend to survive and, interestingly, probably have an enhanced ability to mutate to resistance (Proc. Natl. Acad. Sci. USA, 92, 11736-11740 (1995); J. Bacteriol., 179, 6688-6691 (1997); and Antimicrob. Agents Chemother., 44, 1771-1777 (2000)).

In the light of the above, a new approach to combating the problem of bacterial resistance might be to select and develop antimicrobial agents on the basis of their ability to kill “latent” microorganisms. The production of such agents would allow, amongst other things, for the shortening of chemotherapy regimes in the treatment of microbial infections, thus reducing the frequency with which genotypical resistance arises in microorganisms.

International Patent Application published as WO2012032360 discloses a combination comprising at least one compound selected from the group consisting of an α-adrenergic antagonist, an anthelmintic agent, an antifungal agent, an antimalarial agent, an antineoplastic agent, an antipsychotic agent, an antioxidant, a vasodilator, a vitamin, or a pharmaceutically acceptable derivative thereof; and an antimicrobial compound, together with the use of this combination for killing multiplying, non-multiplying and/or clinically latent microorganisms associated with a microbial infection. Given the importance of antimicrobial agents such as polymyxins in the fight against bacterial infection, the specific identification of agents capable of enhancing their anti-bacterial activity addresses an important need.

International Patent Application, Publication Number WO2000028074 describes a method of screening compounds to determine their ability to kill log phase (i.e. multiplying) and/or clinically latent microorganisms. Using this method, the Applicant has observed that many compounds, such as metergoline, proglumide, sertraline, hydroflumethiazide, perphenazine, fluphenazine and pimethixene or pharmaceutically acceptable derivatives thereof, have a synergistic effect with certain antimicrobial agents such as a polymyxin, against multiplying and/or clinically latent microorganisms.

The present invention is based on the unexpected finding that the combination of at least one compound selected from metergoline, proglumide, sertraline, hydroflumethiazide, perphenazine, fluphenazine and pimethixene or a pharmaceutically acceptable derivative thereof, and a polymyxin selected from polymyxin B and colistin or a pharmaceutically acceptable derivative thereof exhibits synergistic antimicrobial activity against log phase (i.e. multiplying) and/or clinically latent microorganisms. Particularly against log phase bacteria. In other words, the combination has a greater biological activity than the expected additive effect of each agent at the stated dosage level. The surprising biological activity of the combinations of the present invention offers the opportunity to shorten chemotherapy regimens and may result in a reduction in the emergence of microbial resistance associated with the use of such combinations.

Synergy in the context of antimicrobial drugs is measured in a number of ways that conform to the generally accepted opinion that “synergy is an effect greater than additive”. One of the ways to assess whether synergy has been observed is to use the “chequerboard” technique. This is a well-accepted method that leads to the generation of a value called the fractional inhibitory concentration index (FICI). Orhan et al J. Clin. Microbiol. 2005, 43(1):140 describes the chequerboard method and analysis in the paragraph bridging pages 140-141, and explains that the FICI value is a ratio of the sum of the MIC (Minimum Inhibitory Concentration) level of each individual component alone and in the mixture. The combination is considered synergistic when the ΣFIC≤0.5, indifferent when the ΣFIC is >0.5 to <4.0, and antagonistic when the ΣFIC is ≥4.0.

Another accepted test for ascertaining the presence or absence of synergy is to use time-kill methods where the dynamic effect of a drug combination is compared to each drug alone when assessing the effect on bacterial log or stationary-growth over time. Again, the possible results are for synergistic, additive or antagonistic effects.

### SUMMARY OF THE INVENTION

Thus, in one embodiment the present invention provides a combination of at least one compound selected from metergoline, proglumide, sertraline, hydroflumethiazide, perphenazine, fluphenazine and pimethixene, or a pharmaceutically acceptable derivative thereof and a polymyxin selected from polymyxin B and colistin or a pharmaceutically acceptable derivative thereof. Preferably the compound is selected from metergoline, proglumide, sertraline, hydroflumethiazide, and pimethixene, or a pharmaceutically acceptable derivative thereof.

The invention thus provides:

a combination of metergoline or a pharmaceutically acceptable derivative thereof and a polymyxin selected from polymyxin B and colistin, or a pharmaceutically acceptable derivative thereof (e.g. colistin, colistin sulfate or colistimethate sodium);

a combination of proglumide or a pharmaceutically acceptable derivative thereof and a polymyxin selected from polymyxin B and colistin, or a pharmaceutically acceptable derivative thereof (e.g. colistin, colistin sulfate or colistimethate sodium);

a combination of sertraline or a pharmaceutically acceptable derivative thereof (e.g. sertraline hydrochloride) and a polymyxin selected from polymyxin B and colistin, or a pharmaceutically acceptable derivative thereof (e.g. colistin, colistin sulfate or colistimethate sodium);

a combination of hydroflumethiazide or a pharmaceutically acceptable derivative thereof and a polymyxin selected from polymyxin B and colistin, or a pharmaceutically acceptable derivative thereof (e.g. colistin, colistin sulfate or colistimethate sodium);

a combination of perphenazine or a pharmaceutically acceptable derivative thereof and a polymyxin selected from polymyxin B and colistin, or a pharmaceutically acceptable derivative thereof (e.g. colistin, colistin sulfate or colistimethate sodium);

a combination of fluphenazine or a pharmaceutically acceptable derivative thereof (e.g. fluphenazine hydrochloride) and a polymyxin selected from polymyxin B and colistin, or a pharmaceutically acceptable derivative thereof (e.g. colistin, colistin sulfate or colistimethate sodium); and

a combination of pimethixene or a pharmaceutically acceptable derivative thereof (e.g. pimethixene maleate) and a polymyxin selected from polymyxin B and colistin, or a pharmaceutically acceptable derivative thereof (e.g. colistin, colistin sulfate or colistimethate sodium).

In another embodiment the present invention provides the use of the combination defined hereinabove, in the manufacture of a medicament for treating a microbial infection.

Additionally the present invention provides the combination defined hereinabove, for use in the treatment of a microbial infection, preferably a bacterial infection.

In a further embodiment, the invention provides a method of treating a microbial infection which comprises administering to a mammal, including man, the combination defined hereinabove.

There is also provided a pharmaceutical composition comprising at least one compound selected from metergoline, proglumide, sertraline, hydroflumethiazide, perphenazine, fluphenazine and pimethixene, or a pharmaceutically acceptable derivative thereof in combination with a polymyxin selected from colistin or polymyxin B or a pharmaceutically acceptable derivative thereof, and a pharmaceutically acceptable adjuvant, diluent or carrier. In one embodiment the pharmaceutical composition is for use in the treatment of a microbial infection, preferably wherein the microbial infection is a bacterial infection.

In a further embodiment, the invention relates to a product comprising at least one compound selected from metergoline, proglumide, sertraline, hydroflumethiazide, perphenazine, fluphenazine and pimethixene, or a pharmaceutically acceptable derivative thereof in combination with a polymyxin selected from colistin, polymyxin B or a pharmaceutically acceptable derivative thereof, as a combined preparation for simultaneous, separate or sequential use in killing multiplying and/or clinically latent microorganisms associated with a microbial infection. Preferably for killing multiplying bacteria associated with a bacterial infection.

### BRIEF DESCRIPTION OF THE DRAWINGS

FIG. 1 is a time-kill curve (Log CFU/ml against time (hours)) for colistin in the form of colistimethate sodium at a concentration of 16 mg/I, perphenazine at a concentration of 128 mg/I and the combination of colistin (16 mg/I) and perphenazine (128 mg/I).

### DETAILED DESCRIPTION OF THE INVENTION

As described below, the combinations of the present invention have been demonstrated to be particularly effective against drug-resistant bacteria, particularly drug-resistant Gram-negative bacteria, opening the way for said combinations to be administered both to drug-resistant strains and in said strains before drug-resistance is built up, i.e. as a first line treatment.

As used herein, the term “in combination with” covers both separate and sequential administration of the compound and the polymyxin. When the compound and polymyxin are administered sequentially, either the compound or the polymyxin may be administered first. When administration is simultaneous, the compound and polymyxin may be administered either in the same or a different pharmaceutical composition. Adjunctive therapy, i.e. where one agent is used as a primary treatment and the other agent is used to assist that primary treatment, is also an embodiment of the present invention.

The combinations of the present invention may be used to treat microbial infections. In particular they may be used to kill multiplying and/or clinically latent microorganisms associated with microbial infections, preferably multiplying microorganisms associated with microbial infections, e.g. multiplying bacteria associated with Gram-negative bacterial infections. References herein to the treatment of a microbial infection therefore include killing multiplying and/or clinically latent microorganisms associated with such infections.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection.

In one embodiment of the invention, one or more of the aforementioned combinations is used to treat a bacterial infection, in particular the combinations may be used to kill clinically latent microorganisms associated with a bacterial infection. As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept dysgalactiae dysgalactiae, Strept dysgalactiae equisimilis, Strept equi equi, Strept equi zooepidemicus, Strept. iniae, Strept. porcinus and Strept pyogenes), microaerophilic, pyogenic streptococci (Streptococcus “milleri”, such as Strept. anginosus, Strept constellatus constellatus, Strept constellatus pharyngidis and Strept intermedius), oral streptococci of the “mitis” (alpha-haemolytic—Streptococcus “viridans”, such as Strept. mitis, Strept. oralis, Strept. sanguinis, Strept. cristatus, Strept gordonii and Strept. parasanguinis), “salivarius” (non-haemolytic, such as Strept salivarius and Strept vestibularis) and “mutans” (tooth-surface streptococci, such as Strept. criceti, Strept. mutans, Strept ratti and Strept. sobrinus) groups, Strept. acidominimus, Strept. bovis, Strept. faecalis, Strept. equinus, Strept pneumoniae and Strept. suis, or Streptococci alternatively classified as Group A, B, C, D, E, G, L, P, U or V Streptococcus);

Gram-negative cocci, such as Neisseria gonorrhoeae, Neisseria meningitidis, Neisseria cinerea, Neisseria elongate, Neisseria flavescens, Neisseria lactamica, Neisseria mucosa, Neisseria sicca, Neisseria subflava and Neisseria weaveri; Bacillaceae, such as Bacillus anthracis, Bacillus subtilis, Bacillus thuringiensis, Bacillus stearothermophilus and Bacillus cereus; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. Iwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. camis, C. celatum, C. clostridioforme, C. cochlearium, C. cocleatum, C. fallax, C. ghonfi, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium mucogenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter lari and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonfi, Borrelia bissettii, Borrelia garinfi, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gaffinarum, Pasteurella haemolytica, Pasteurella multocida multocida, Pasteurella multocida gafficida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetii); Legionella (e.g. Legionalla anisa, Legionalla birminghamensis, Legionalla bozeman II, Legionalla cincinnatiensis, Legionalla dumoffii, Legionalla feelefi, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia cepacia; Burkholderia mallei and Burkholderia pseudomallei; Francisella tularensis; Gardnerella (e.g. Gardneralla vaginalis and Gardneralla mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella baciffiformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillium (e.g. Spirillum minus); Bacteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canons, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. felis, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); and Ureaplasma (e.g. Ureaplasma parvum and Ureaplasma urealyticum).

Preferably, the bacterial infections treated by the combinations described herein are gram-negative bacterial infections. Particular Gram-negative bacteria that may be treated using a combination of the invention include:

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); Haemophilis influenzae; Mycobacteria, such as Mycobacterium tuberculosis; and Enterobacter (e.g. Enterobacter cloacae).

Preferably, the bacteria are Enterobacteriaceae, such as Escherichia coli, Klebsiella (e.g. Klebs. pneumoniae and Klebs. oxytoca) and Enterobacter (e.g. Enterobacter cloacae). Particularly preferred are Escherichia coli, and Klebsiella, e.g. Escherichia coli, and Klebs. pneumoniae including Klebs. pneumoniae subsp. pneumoniae.

The combination of the present invention is particularly beneficial in treating (multi)-drug-resistant ((M)DR) bacteria. With respect to Enterobacteriaceae, drug resistance most often builds up to carbapenemase i.e. carbapenemase-resistant strains and “extended spectrum β-lactamase” (ESBL) strains for example New Delhi Metallo-beta-lactamase-1 (NDM-1) resistant Klebs. Pneumonia, and NDM-1 E. coli.

It should be kept in mind that although a combination such as that claimed may initially be demonstrated to be functional in treating (M)DR strains, they can then be used in treating non-resistant strains. This is especially valuable in the context of the presently claimed combination where the primary therapy for Enterobacteriaceae, such as Escherichia coli, and Klebsiella (e.g. Klebs. pneumoniae and Klebs. oxytoca) are anti-microbial drugs that are expensive due to prevailing patent protection. The replacement of such “ethical” drugs by a combination of “generic” antibiotics is thought to be beneficial from a therapeutic perspective as well as financial/economic perspective in times where governments are seeking to reduce the cost of healthcare.

The combinations of the present invention may be used to treat infections associated with any of the above-mentioned bacterial organisms, and in particular they may be used for killing multiplying and/or clinically latent microorganisms associated with such an infection, e.g. a Gram-negative bacterial infection.

Particular conditions which may be treated using the combination of the present invention include those which are caused by Gram-negative bacteria such as abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, Gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

It will be appreciated that references herein to “treatment” extend to prophylaxis as well as the treatment of established diseases or symptoms.

As used herein the term “pharmaceutically acceptable derivative” means:

(a) pharmaceutically acceptable salts; and/or

(b) solvates (including hydrates).

Suitable acid addition salts include carbon/late salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts, and the like.

The polymyxin is colistin or polymyxin B or a pharmaceutically acceptable derivative thereof. For example, colistin sulfate, colistimethate sodium, colistin sodium methanesulfonate, or polymyxin B sulfate. Particularly preferred is colistin, colistin sulfate, colistin sodium methane sulfonate or colistimethate sodium, e.g. colistin or colistimethate sodium.

Colistin is also known as polymyxin E and is an antibiotic produced by certain strains of the bacteria Paenibacillus polymyxa. Colistin has the following chemical structure and the IUPAC chemical name N-(4-amino-1-(1-(4-amino-1-oxo-1-(3,12,23-tris(2-aminoethyl)-20-(1-hydroxyethyl)-6,9-diisobutyl-2,5,8,11,14,19,22-heptaoxo-1,4,7,10,13,18-exaazacyclotricosan-15-ylamino)butan-2-ylamino)-3-hydroxybutan-2-ylamino)-1-oxobutan-2-yl)-N,5-dimethylheptanamide.

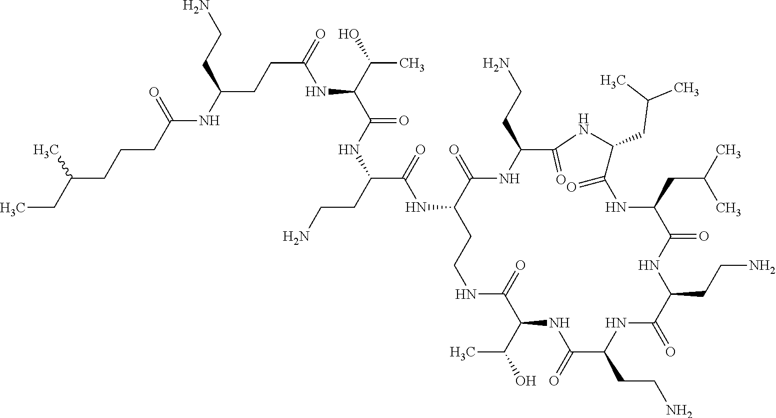 [SMILES: CCC(C)CCCC(=O)N[C@@H](CCN)CCC(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

Metergoline is a psychoactive drug of the ergoline chemical class. It is also known as {[(8β)-1,6-dimethylergolin-8-yl]methyl}carbamate and has the following chemical structure:

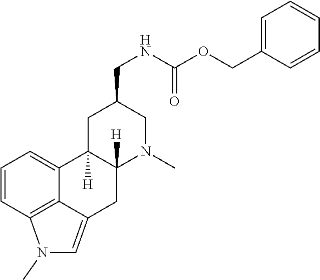

Proglumide is a drug that is known to inhibit gastrointestinal motility and reduce gastric secretions. It is commercially available under the brand name Milid and is known by the chemical name of 4-benzamido-5-(dipropylamino)-5-oxopentanoic acid and with the following chemical structure:

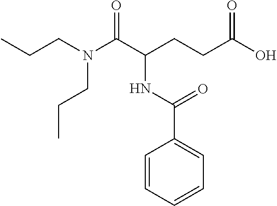 [SMILES: CCCN(CCC)C(=O)C(CCC(=O)O)NC(=O)c1ccccc1]

Proglumide may be used in the form of its sodium salt. In the present invention it is preferably in the non-salt form, i.e. as proglumide.

Sertraline is an antidepressant of the selective serotonin reuptake inhibitor (SSRI) class. It is commercially available under the brand name Zoloft or Lustral®. Sertraline is also known as (1S,4S)-4-(3,4-dichlorophenyl)-N-methyl-1,2,3,4-tetrahydronaphthalen-1-amine with the following chemical structure:

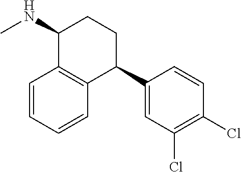 [SMILES: CN[C@H]1CC[C@@H](c2ccc(Cl)c(Cl)c2)c2ccccc21]

Sertraline may be used in the form of its hydrochloride salt. In the present invention it is preferably in the salt form, i.e. as sertraline hydrochloride.

Hydroflumethiazide is a diuretic which is commercially available under the brand name Aldactide. It has the chemical name of 1,1-Dioxo-6-(trifluoromethyl)-3,4-dihydro-2H-1,2,4-benzothiadiazine-7-sulfonamide and the following chemical structure:

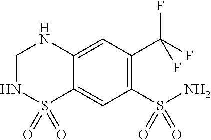 [SMILES: NS(=O)(=O)c1cc2c(cc1C(F)(F)F)NCNS2(=O)=O]

Perphenazine is an antipsychotic drug which is chemically classified as piperazinyl phenothiazine and commercially available under the brand name Trilafon, Fentazin or Triptafen. It has the chemical name of 2-[4-[3-(2-chloro-10H-phenothiazin-10-yl) propyl]piperazin-1-yl]ethanol and the following chemical structure:

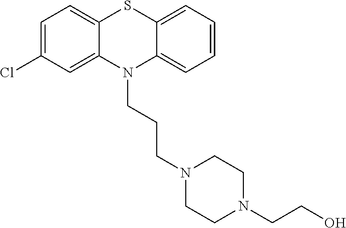 [SMILES: OCCN1CCN(CCCN2c3ccccc3Sc3ccc(Cl)cc32)CC1]

Perphenazine may be used in the form of its sulfoxide salt. In the present invention it is preferably in the non-salt form, i.e. as perphenazine.

Fluphenazine is an antipsychotic drug which is commercially available under the brand name Prolixin or Modecate among others. It is known by the chemical name of 2-[4-[3-[2-(trifluoromethyl)-10H-phenothiazin-10-yl]propyl]piperazin-1-yl]ethanol, and has the following structure:

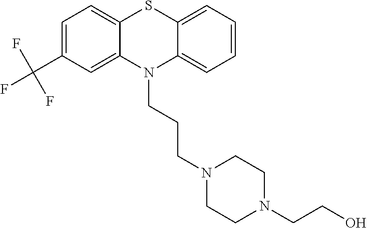 [SMILES: OCCN1CCN(CCCN2c3ccccc3Sc3ccc(C(F)(F)F)cc32)CC1]

Fluphenazine may be used in the form of its hydrochloride salt, its dihydrochloride salt, its decanoate salt, its decanoate hydrochloride salt, its enantate salt, its enanthate dihydrochloride salt, or its octanoate salt. In the present invention it is preferably in the hydrochloride salt form, i.e. as fluphenazine hydrochloride.

Pimethixene is an antihistamine and anticholinergic of the thioxanthene chemical class that is often used to treat hyperactivity, anxiety, sleep disorders and allergy. It is also used for anesthesia and as a bronchodilator. Pimethixene is also known as 1-methyl-4-(9H-thioxanthen-9-ylidene)piperidine and has the following chemical structure:

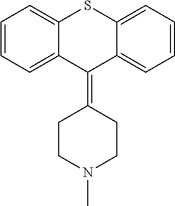 [SMILES: CN1CCC(=C2c3ccccc3Sc3ccccc32)CC1]

Pimethixene may be used in the form of its maleate salt. In the present invention it is preferably in salt form, i.e. as pimethixene maleate.

Compounds for use according to the invention may be administered as the raw material but the active ingredients are preferably provided in the form of pharmaceutical compositions. The active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation or insufflation administration. The most suitable route of administration may depend upon the condition and disorder of the patient. Preferably, the compositions of the invention are formulated for oral administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Formulations suitable for oral administration may be presented as discrete units such as capsules, cachets or tablets (e.g. chewable tablets in particular for paediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

A tablet may be made by compression or moulding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Moulded tablets may be made by moulding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Combinations for use according to the invention may be presented in a pack or dispenser device which may contain one or more unit dosage forms containing the active ingredients. The pack may, e.g. comprise metal or plastic foil, such as a blister pack. Where the compositions are intended for administration as two separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The compounds for use in the present invention are commercially available and/or may be prepared using conventional methods known in the art.

Suitable dosages and formulations for the administration of colistin and pharmaceutically acceptable derivatives thereof are known in the art. A suitable dosage and formulation for colistin sulfate is for instance described in the product label for Colomycin® which can be found at http://www.medicines.org.uk/emc/medicine/6301/SPC/Colomycin+Tablets/. A suitable dosage and formulation for colistimethate sodium is described in the product label for colistimethate sodium 1. Million I.U. Powder for Solution for Injection which can be found at https://www.medicines.org.uk/emc/medicine/23413.

Suitable dosages and formulations for the administration of metergoline, proglumide, sertraline, hydroflumethiazide, perphenazine, fluphenazine and pimethixene or a pharmaceutically acceptable derivative thereof are also known in the art.

A suitable dosage and formulation for sertraline hydrochloride is for instance described in the product label for Lustral® which can be found at https://www.medicines.org.uk/emc/medicine/27116 or in the product label for generic Sertraline which can be found at https://www.medicines.org.uk/emc/medicine/31858. A suitable dosage and formulation for hydroflumethiazide is described in the product label for Aldactide which can be found at https://www.medicines.org.uk/emc/medicine/10703. A suitable dosage and formulation for perphenazine is described in the product label for Fentazin which can be found at https://www.medicines.org.uk/emc/medicine/22596.

The administration of the combination of the invention by means of a single patient pack, or patient packs of each composition, including a package insert directing the patient to the correct use of the invention is a desirable feature of this invention.

According to a further embodiment of the present invention there is provided a patient pack comprising at least one active ingredient of the combination according to the invention and an information insert containing directions on the use of the combination of the invention.

In another embodiment of the invention, there is provided a double pack comprising in association for separate administration, an antimicrobial agent, preferably having biological activity against clinically latent microorganisms, and one or more of the compounds disclosed herein preferably having biological activity against clinically latent microorganisms.

The amount of active ingredients required for use in treatment will vary with the nature of the condition being treated and the age and condition of the patient, and will ultimately be at the discretion of the attendant physician or veterinarian. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three, four or more sub-doses per day.

Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationery phase;
(2) treating the stationery phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO 2005014585, the disclosures of which document are hereby incorporated by reference), of Minimum Inhibitory Concentration (“MIC”) or Minimum Bactericidal Concentration (“MBC”) for a test compound. Specific examples of such methods are described below.

### EXAMPLES

### Example 1: In Vitro Synergistic Effect of Hydroflumethiazide (HT0120443) and Colistin (Colistimethate Sodium) Against Log Phase NDM-1 Escherichia coli Using the Chequerboard Method

Log phase growth of NDM-1 Escherichia coli was carried out as described in the art. Hydroflumethiazide and colistin (colistimethate sodium) were obtained from commercial sources (e.g. Sigma Aldrich® UK).

The effects of each combination of the present invention were examined by using the chequerboard method (as described in Antimicrob Chemo (2013) 68, 374-384), and calculating the fractional inhibitory concentration index (FICI) of each combination, as follows: (MIC of drug A, tested in combination)/(MIC of drug A, tested alone)+(MIC of drug B, tested in combination)/(MIC of drug B, tested alone). The interaction of the combination was defined as showing synergy if ΣFIC is 0.5, indifferent when the ΣFIC is >0.5 to <4.0, and antagonistic when the ΣFIC is ≥4.0.

The chequerboard data is shown below:

The FICI was equal to 0.06 indicating that colistin (colistimethate sodium) and hydroflumethiazide exhibited a synergistic effect against log phase NDM-1 E. coli.

### Example 2: In Vitro Synergistic Effect of Perphenazine (HT0120789) and Colistin (Colistimethate Sodium) Against Log Phase NDM-1 Escherichia coli Using the Chequerboard Method

Log phase growth of NDM-1 Escherichia coli was carried out as described in the art. Perphenazine and colistimethate sodium were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.31 indicating that colistin (colistimethate sodium) and perphenazine exhibited a synergistic effect against log phase NDM-1 E. coli.

### Example 3: In Vitro Synergistic Effect of Metergoline (HT0121504) and Colistin (Colistimethate Sodium) Against Log Phase NDM-1 Escherichia coli Using the Chequerboard Method

Log phase growth of NDM-1 Escherichia coli was carried out as described in the art. Metergoline and colistimethate sodium were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

The FICI was equal to 0.141 indicating that colistin (colistimethate sodium) and metergoline exhibited a synergistic effect against log phase NDM-1 E. coli.

### Example 4: In Vitro Synergistic Effect of Colistin (Colistimethate Sodium) and Proglumide (HT0120075) Against Log Phase NDM-1 Klebsiella pneumoniae Using the Chequerboard Method

Log phase growth of NDM-1 K. pneumoniae was carried out as described in the art. Colistimethate sodium and proglumide were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.094 indicating that colistimethate sodium and proglumide exhibited a synergistic effect against log phase NDM-1 K. pneumoniae.

### Example 5: In Vitro Synergistic Effect of Colistin (Colistimethate Sodium) and Fluphenazine Hydrochloride (HT0120633) Against Log Phase NDM-1 Klebsiella pneumoniae Using the Chequerboard Method

Log phase growth of NDM-1 K. pneumoniae was carried out as described in the art. Colistimethate sodium and fluphenazine hydrochloride were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.133 or 0.127 indicating that colistin (colistimethate sodium) and fluphenazine hydrochloride exhibited a synergistic effect against log phase NDM-1 K. pneumoniae.

### Example 7: In Vitro Synergistic Effect of Colistin (Colistimethate Sodium) and Sertraline Hydrochloride (HT0120854) Against Log Phase NDM-1 Klebsiella pneumoniae Using the Chequerboard Method

Log phase growth of NDM-1 K. pneumoniae was carried out as described in the art. Colistimethate sodium and sertraline hydrochloride were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.16 indicating that colistimethate sodium and sertraline hydrochloride exhibited a synergistic effect against log phase NDM-1 K. pneumoniae.

### Example 8: In Vitro Synergistic Effect of Colistin (Colistimethate Sodium) and Pimethixene Maleate (HT0121290) Against Log Phase NDM-1 Klebsiella pneumoniae Using the Chequerboard Method

Log phase growth of NDM-1 K. pneumoniae was carried out as described in the art. Colistimethate sodium and pimethixene maleate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.25, 0.141 or 0.094 indicating that colistimethate sodium and pimethixene maleate exhibited a synergistic effect against log phase NDM-1 K. pneumoniae.

### Example 9: In Vitro Synergistic Effect of Colistimethate Sodium and Fluphenazine Hydrochloride (HT0120633) Against Log Phase NDM-1 Klebsiella pneumoniae Using the Chequerboard Method

Log phase growth of NDM-1 K. pneumoniae was carried out as described in the art. Colistimethate sodium and fluphenazine hydrochloride were obtained from commercial sources (e.g. Sigma Aldrich® UK). The effects of each combination of the present invention were examined by using the chequerboard method and calculating the fractional inhibitory concentration index (FICI) of each combination in the same manner as for Example 1.

The chequerboard data is shown below:

The FICI was equal to 0.133 or 0.127 indicating that colistin (colistimethate sodium) and fluphenazine hydrochloride exhibited a synergistic effect against log phase NDM-1 K. pneumoniae.

### Example 10: In Vitro Synergistic Effect of Perphenazine in Combination with Colistimethate Sodium Against Log Phase NDM-1 Klebsiella Pneumoniae

The objective of this example was to test the synergistic effect of perphenazine and colistin (in the form of colistimethate sodium) in combination against log phase NDM-1 Klebsiella Pneumoniae by time-kill methods over a time period of 24 hours. As described hereinabove, time-kill methods are another accepted test for ascertaining the presence or absence of synergy, and involve comparing the dynamic effect of a drug combination with each drug alone when assessing the effect on bacterial log or stationary-growth over time. The results can either show that the drug combination is synergistic, additive or antagonistic.

Materials and Methods
1. Bacterial strain used: BAA2473 strain of Klebsiella pneumoniae
2. Growth of bacteria: Log phase growth of BAA2473 was carried out according to known methods in the art, e.g. SOP R-005-00 Log Phase Growth of Bacteria.
3. Compound preparation:
   i. Perphenazine was ordered from Sigma Aldrich® UK.
   ii. Colistin was obtained as Colistimethate sodium (Forest Laboratories UK 1 million I. U. 80 mg).
4. The overnight culture was diluted with nutrient broth (Oxoid) to 107 CFU/ml and 280 μl and 290 ul of the culture was added to each combination well and drug respectively, to make the final concentration of 300 μl.
5. Incubation of the compounds with the bacterial suspension was carried out for 24 hours. At 0, 2, 4, 7 and 24 hours, CFU counts were performed to measure the kill effects of the drug combination.

Results and Discussion

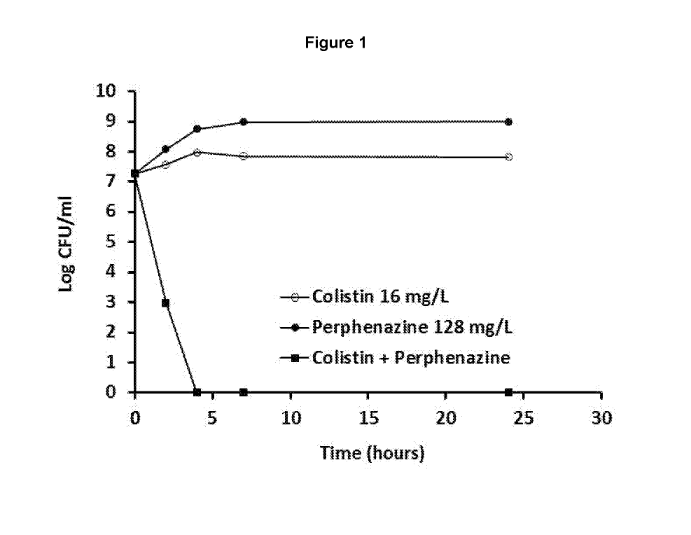

The results are displayed in FIG. 1 where it can be seen that both colistin at a concentration of 16 mg/L and perphenazine at a concentration of 128 mg/L had little or no effect against NDM-1 K. pneumoniae. When used in combination, however, a significant synergistic effect can be seen. FIG. 1 demonstrates a complete kill of bacteria at 4 hours when perphenazine and colistin are used in combination.

In view of the lack of activity for each of the drugs alone against NDM-1 K. pneumoniae, the synergy seen for the combination of the claimed invention is a surprising and advantageous technical effect for the treatment of microbial infections.

1. A combination comprising: (i) at least one compound selected from metergoline, proglumide, sertraline, hydroflumethiazide, perphenazine, fluphenazine and pimethixene, or a pharmaceutically acceptable derivative thereof; and (ii) a polymyxin selected from polymyxin B and colistin or a pharmaceutically acceptable derivative thereof.

2. The combination according to claim 1, wherein the polymyxin is colistin or colistimethate sodium.

3. The combination according to claim 1, wherein the combination comprises at least one compound selected from metergoline, proglumide, sertraline, hydroflumethiazide and pimethixene or a pharmaceutically acceptable derivative thereof.

4. (canceled)

5. The method according to claim 16, wherein the microbial infection is a bacterial infection.

6. The method according to claim 5, wherein the bacterial infection is caused by E. coli or Klebs. pneumoniae.

7. The method according to claim 5, wherein the infection is caused by a drug-resistant strain of the bacteria.

8. (canceled)

9. The combination according to claim 13, wherein polymyxin is colistin or colistimethate sodium.

10. The combination according to claim 1, wherein the combination is used to treat a microbial infection.

11. The combination according to claim 17, wherein the bacterial infection is a gram-negative bacterial infection caused by E. coli, or Klebs. pneumoniae.

12. The combination according to claim 11, wherein the infection is caused by a drug-resistant strain.

13. The combination according to claim 1 for use in the treatment of abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, Gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

14. A pharmaceutical composition comprising: (a) at least one compound selected from metergoline, proglumide, sertraline, hydroflumethiazide, perphenazine, fluphenazine and pimethixene, or a pharmaceutically acceptable derivative thereof, in combination with: (b) a polymyxin selected from polymyxin B and colistin, or a pharmaceutically acceptable derivative thereof; and (c) and a pharmaceutically acceptable adjuvant, diluent or carrier.

15. A product comprising: (i) at least one compound selected from metergoline, proglumide, sertraline, hydroflumethiazide, perphenazine, fluphenazine and pimethixene, or a pharmaceutically acceptable derivative thereof, in combination with; and (ii) a polymyxin selected from colistin or polymyxin B, wherein the product is a combined preparation for the simultaneous, separate or sequential use in treating a microbial infection.

16. A method of treating a microbial infection, the method comprising the step of administering at least one therapeutic amount of the combination according to claim 1 to a desired subject.

17. The method according to claim 16, wherein the method is used in the treatment of abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, Gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

18. The combination of claim 10, wherein the microbial infection is a bacterial infection.

19. The combination according to claim 10, wherein the infection is caused by a drug-resistant strain.
